(19) United States
(12) Patent Application Publication
(10) Pub. No.: US 2023/0218431 A1
(43) Pub. Date: Jul. 13, 2023

(54) CORE TEMPERATURE COOLING SYSTEM

(71) Applicant: Nathan Daniel Brandt, Murrieta, CA (US)

(72) Inventor: Nathan Daniel Brandt, Murrieta, CA (US)

(21) Appl. No.: 18/155,021

(22) Filed: Jan. 16, 2023

Related U.S. Application Data
(60) Provisional application No. 63/279,778, filed on Nov. 16, 2021.

(51) Int. Cl. A61F 7/02

(52) U.S. Cl. CPC A61F 7/02; A61F 2007/0063

(57) ABSTRACT

Embodiments of the present disclosure may include an apparel device, including an apparel body including an outer layer and an inner layer, and an interior configured to receive a body part of a user. Embodiments may also include a heat transfer panel disposed between the outer layer and the inner layer. In some embodiments, the heat transfer panel includes a first side and a second side, the first side disposed adjacent the interior of the apparel body. Embodiments may also include a compressed fluid source in fluid communication with the second side of the heat transfer panel. In some embodiments, the compressed fluid source may be configured to selectively deliver compressed fluid to the heat transfer panel. In some embodiments, the heat transfer panel may be configured to cool the interior of the apparel body upon delivery of the compressed fluid to the heat transfer panel.

### CROSS-REFERENCE TO RELATED APPLICATIONS

This application claims the benefit of U.S. Provisional Application No. 63/279,778, filed on Nov. 16, 2021. The entire disclosure of the above application is incorporated herein by reference.

### FIELD

The present disclosure relates to cooling devices, and more specifically, to a device, system, and method of cooling the body.

### INTRODUCTION

The statements in this section merely provide background information related to the present disclosure and may not constitute prior art.

The human body may go to great lengths to maintain an internal body temperature within a range. When it is cold, the body may shiver and when it is too hot, the body may produce sweat. Dehydration and loss of essential electrolytes may lead to lower than optimal body function. A high-profile example is Lebron James, who experienced muscle cramps when playing in hotter than normal conditions, and for example, was unable to continue playing during an important game in the 2014 NBA finals. While athletes across sports may be affected by heat, performance and productivity may decrease for anyone in the heat. A core body temperature must be maintained within a safe range so that other body systems are not put at risk.

An important co-enzyme in the process that turns adenosine triphosphate (ATP) to adenosine diphosphate (ADP), the process that gives muscle cells energy to move and constrict, is heat sensitive and may be less active as the body temperature increases. In fact, this co-enzyme may become inactive as body temperature approaches 104 degrees as a method of protecting the body.

Over the years various mechanisms have been used to maintain a cooled internal body temperature. These mechanisms may include sweat wicking fabric, breathable clothing, personal cooling misters, and fans. However, it is not easy to lower the core temperature of the body, particularly while the body is moving.

Accordingly, there is a continuing need for a cooling system capable of lowering the core body temperature during activities that may raise the body temperature and when the body is exposed to heat.

### SUMMARY

In concordance with the present disclosure, a cooling system capable of lowering the core body temperature during activities that may raise the body temperature and when the body is exposed to heat, is surprisingly discovered.

Various embodiments of the present technology may include articles of manufacture, systems, and processes that relate to apparel and devices for cooling a body. It has been discovered that certain areas of the body may function as a heat exchange zone, such as an arteriovenous anastomoses (AVA) zone, which allows the body to exchange heat with its environment at a faster rate. These AVA zones may be located on the face, palms, and soles of the feet. By applying a cooling effect to these AVA zones, the body may be cooled. In particular, embodiments of the present invention may be used by athletes, non-athletes, and other persons to cool the body. In certain embodiments, the present technology may be used as a way to reduce swelling, inflammation, and to provide comfort to a user.

Embodiments of the present disclosure may include an apparel device. The apparel device may include an apparel body including an outer layer, an inner layer, and an interior configured to receive a body part of a user. Embodiments may also include a heat transfer panel disposed between the outer layer and the inner layer. In some embodiments, the heat transfer panel may include a first side and a second side, the first side disposed adjacent the interior of the apparel body.

Embodiments may also include a compressed fluid source in fluid communication with the second side of the heat transfer panel. In some embodiments, the compressed fluid source may be configured to selectively deliver compressed fluid to the heat transfer panel. The heat transfer panel may be configured to cool the interior of the apparel body upon delivery of the compressed fluid to the heat transfer panel. Embodiments may also include a vent disposed within the apparel body. The vent may be configured to expel the compressed fluid and/or heat to an environment outside of the apparel body following the delivery of the compressed fluid to the heat transfer panel.

In some embodiments, the apparel device may include a valve in fluid communication with the compressed fluid source and the heat transfer panel. The valve may be configured to selectively deliver the compressed fluid from the compressed fluid source to the heat transfer panel. In some embodiments, the valve may be a mechanical valve and may be automatically opened when a predetermined amount of pressure may be applied to the apparel body. The valve may be configured to be manually opened by the user. In some embodiments, the apparel device may include a controller. The valve may be an electronic valve in electronic communication with the controller. In some embodiments, the apparel device may include a sensor in electronic communication with the controller.

The sensor may include at least one of a temperature sensor and a pressure sensor, the temperature sensor may be configured to detect an internal temperature within an interior of the apparel body and transmit a signal representative of the internal temperature to the controller. The controller may be configured to actuate the electronic valve to deliver a flow of the compressed fluid to the heat transfer panel upon the internal temperature exceeding a predetermined temperature. In certain embodiments, once the internal temperature no longer exceeds the predetermined temperature, the controller may actuate the electronic valve to cease the flow of the compressed fluid to the heat transfer panel. The pressure sensor may be configured to detect an amount of the compressed fluid within the compressed fluid source.

The compressed fluid source may include a compressed fluid cannister. The compressed fluid cannister may be disposed within a recess of the apparel body. In some embodiments, the compressed fluid may be rechargeable based on a movement of the user while wearing the apparel device. In some embodiments, the compressed fluid cannister may be removeable and replaceable when the compressed fluid of the compressed fluid cannister is fully discharged. In some embodiments, the apparel body may be selected from a group consisting of footwear, handwear, headwear, upper body clothing, lower body clothing, and combinations thereof. The compressed fluid may be selected from a group consisting of liquified air, liquified carbon dioxide, a refrigerant, and combinations thereof.

Embodiments of the present disclosure may also include an apparel system including an apparel device. The apparel device may have an apparel body including an outer layer, an inner layer, and an interior configured to receive a body part of a user. A heat transfer panel may be disposed between the outer layer and the inner layer. In some embodiments, the heat transfer panel may include a first side and a second side, the first side disposed adjacent the interior of the apparel body. A compressed fluid source may be in fluid communication with the second side of the heat transfer panel.

The compressed fluid source may be configured to selectively deliver the compressed fluid to the heat transfer panel. In some embodiments, the heat transfer panel may be configured to cool the interior of the apparel body upon delivery of the compressed fluid to the heat transfer panel. A vent may be disposed within the apparel body. The vent may be configured to expel the compressed fluid to an environment outside of the apparel body following the delivery of the compressed fluid to the heat transfer panel. Embodiments may also include a regulator in electronic communication with the apparel device. In some embodiments, the regulator may be configured to permit the user to operate the apparel device. The apparel system may include a controller. The controller may include a transceiver in electronic communication with the regulator. In some embodiments, the regulator includes a user interface configured to permit for at least one of a selective delivery and pre-programmed delivery of the compressed fluid to the heat transfer panel.

Embodiments of the present disclosure may also include a method of cooling a body part of a user. The method may include steps of providing an apparel device, the apparel device having an apparel body including an outer layer and an inner layer, and an interior configured to receive the body part of the user, a heat transfer panel disposed between the outer layer and the inner layer. The heat transfer panel may include a first side and a second side, the first side disposed adjacent the interior of the apparel body. A compressed fluid source may be in fluid communication with the second side of the heat transfer panel.

In some embodiments, the compressed fluid source may be configured to selectively deliver compressed fluid to the heat transfer panel. In some embodiments, the heat transfer panel may be configured to cool the interior of the apparel body upon delivery of the compressed fluid to the heat transfer panel. A vent may be disposed within the apparel body.

In some embodiments, the vent may be configured to expel the compressed fluid to an environment outside of the apparel body following the delivery of the compressed fluid to the heat transfer panel. In certain embodiments, the expelled compressed fluid may have expanded to a gas. In certain embodiments, the expelled compressed fluid may comprise a gas, a liquid, and combinations thereof. Embodiments may also include donning, by the user, the apparel device. Embodiments may also include delivering the compressed fluid from the compressed fluid source to the heat transfer panel, thereby controlling an internal temperature of the apparel device. In some embodiments, the method may include a step of replacing the compressed fluid source upon the compressed fluid source being fully discharged.

In an exemplary embodiment, a cooling system may use the body's natural heat exchange zones to lower body temperature and help maintain temperature homeostasis. When the body temperature is brought down closer to a normal range, this reduces the need for extreme sweating and maintains the body's access to chemical energy, ATP, for performance and recovery. Athletes may keep running, construction workers may keep working and soldiers may keep marching. In certain embodiments, the cooling system may be disposed at least partially within a bottom portion of apparel, such as footwear, for example, the sole of a shoe.

In certain embodiments, the cooling system may include a compression tank that holds compressed gas as a liquid, an expansion tank that holds expanded liquid as gas, a heat transfer plate, a pumping mechanism, and a release mechanism, such as a spray nozzle. The compression tank may be in fluid communication with the expansion tank. The compression tank may be configured to receive gas from the expansion tank and compress the gas causing a phase change turning the gas to liquid. In certain embodiments, a refrigerant may be used to achieve the desired cooling without requiring pressure to compress it from a gas to a liquid. The expansion tank may be configured to receive liquid from the compression tank and rapidly expand the liquid causing a phase change turning the liquid to gas.

The heat transfer plate may be disposed on top of the expansion tank. More specifically, the heat transfer plate may be located between the expansion tank and the sole of a wearer's foot, where the system is disposed in an article of footwear. In one example, the heat transfer plate may form a top of the expansion tank. The heat transfer plate may be positioned such that the heat transfer plate is exposed for direct contact with an AVA zone of the body.

The pumping mechanism may be configured to pump the expanded gas from the expansion tank to the compression tank. The pumping mechanism may include a mechanical or an electrical pumping mechanism. In certain embodiments, the pumping mechanism may be operated by movement and weight of the user. As the user moves, a weight of the user may apply force to the pumping mechanism causing the pumping mechanism to compress thereby compressing the gas into a liquid and pumping the liquid into the compression tank. Alternatively, a refrigerant may be used to achieve the desired cooling without requiring pressure to compress it without requiring pressure to compress it from gas to liquid.

The release mechanism may be configured to release or spray the compressed gas/liquid from the compression tank to the expansion tank. When the compressed gas/liquid is released or sprayed into the expansion tank via the release mechanism, the compressed gas/liquid is thereby dispersed against the heat transfer plate, which is in contact with an AVA zone of the body. The heat transfer plate may be in direct or indirect contact with an AVA zone of the body. In certain embodiments, the heat transfer plate may include a layer or film disposed between the heat transfer plate and the body. The cooling system may include more than one release mechanism. In certain embodiments, the cooling system may include four release mechanisms. As would be understood by someone of ordinary skill in the art, the cooling system may include any number of appropriately desired release mechanisms.

The cooling system may include a one-way valve configured to fluidly connect the expansion tank to the pumping mechanism. Gas may flow from the expansion tank through the one-way valve and into the pumping mechanism where the gas may be compressed into a liquid. The pumping mechanism may include or be attached to a heat sink that transfers the generated heat away from the system. As such, the pumping mechanism pumps the cooled compressed gas/liquid into the compression tank. The release mechanism may be configured to fluidly connect the compression tank and expansion tank and release the compressed gas/liquid into the expansion tank where the compressed gas/liquid is dispersed against the heat transfer plate, thereby cooling the heat transfer plate. The cooled heat transfer plate may be in direct or indirect contact with the AVA zone of the body such that heat is transferred away from the AVA zone of the body of the user.

It has been advantageously found that by using the principals of thermodynamics involved in a phase change that occurs when a rapidly expanding fluid under pressure, such as when a pressurized liquid phases into a gas at a lower pressure, a heat transfer plate in contact with an AVA zone of the body may be rapidly cooled and pull heat from the body.

Further areas of applicability will become apparent from the description provided herein. The description and specific examples in this summary are intended for purposes of illustration only and are not intended to limit the scope of the present disclosure, or to limit the parts of the body that may be cooled.

### DRAWINGS

The drawings described herein are for illustrative purposes only of selected embodiments and not all possible implementations, and are not intended to limit the scope of the present disclosure.

FIG. 1 is a block diagram illustrating an apparel device, according to some embodiments of the present disclosure.

FIG. 2 is a block diagram further illustrating the apparel device from FIG. 1, according to some embodiments of the present disclosure.

FIG. 3 is a block diagram further illustrating the apparel device from FIG. 1, according to some embodiments of the present disclosure.

FIG. 4 is a block diagram illustrating an apparel system, according to some embodiments of the present disclosure.

FIG. 5 is a block diagram further illustrating the apparel system from FIG. 4, according to some embodiments of the present disclosure.

FIG. 6 is a flowchart that describes a method of cooling a body part of a user, according to some embodiments of the present disclosure.

FIG. 7 is a diagram showing a configuration of cooling a body with a compressed fluid source, according to some embodiments of the present disclosure.

FIG. 8 is a diagram showing a second configuration of cooling a body with a compressed fluid source using a thermally conductive fluid and a refrigerant, according to some embodiments of the present disclosure.

FIG. 9 is a front perspective view of an apparel device implemented within an article of footwear, according to some embodiments of the present disclosure.

FIG. 10 is an enlarged plan view of an inlet port for a compressed fluid source, a heat transfer panel and vent of the apparel device implemented within the article of footwear, according to some embodiments of the present disclosure.

FIG. 11 is a cross sectional view showing a compressed fluid path of the apparel device implemented within the article of footwear, according to some embodiments of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Example embodiments of the present technology are provided with reference to the several figures enclosed herewith.

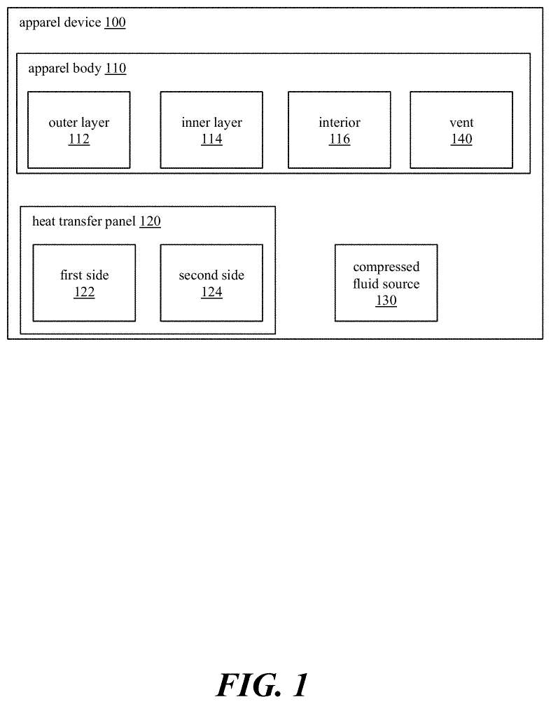

FIG. 1 is a block diagram that describes an apparel device 100, according to some embodiments of the present disclosure. In some embodiments, the apparel device 100 may include an apparel body 110. A heat transfer panel 120 may be disposed between an outer layer 112 and an inner layer 114 of the apparel body 110. A compressed fluid source 130 may be in fluid communication with a second side 124 of the heat transfer panel 120. A vent 140 may be disposed within the apparel body 110. An interior 116 of the apparel body may be configured to receive a body part of a user.

The heat transfer panel 120 may include a first side 122 and a second side 124. The first side 122 may be disposed adjacent the interior 116 of the apparel body 110. The compressed fluid source 130 may be configured to selectively deliver compressed fluid to the heat transfer panel 120. The heat transfer panel 120 may be configured to cool the interior 116 of the apparel body 110 upon delivery of the compressed fluid to the heat transfer panel 120. The vent 140 may be configured to expel the compressed fluid to an environment outside of the apparel body 110 following the delivery of the compressed fluid to the heat transfer panel 120.

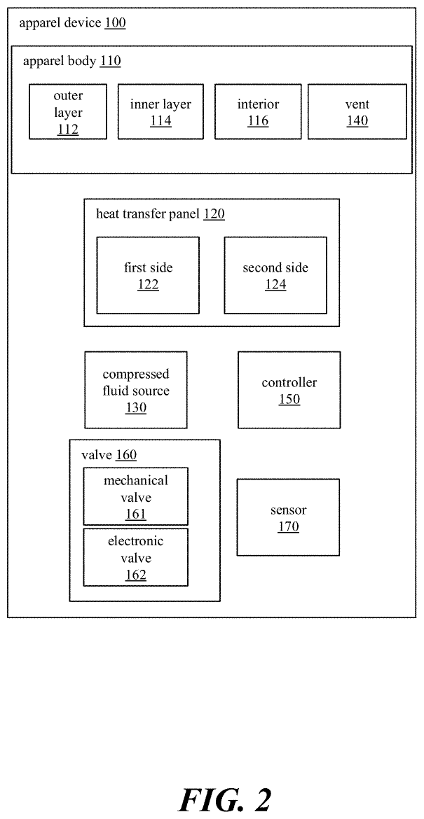

In some embodiments, such as shown in FIG. 2, a valve 160 may be a mechanical valve 161 that is automatically opened when a predetermined amount of pressure is applied to the apparel body 110. In some embodiments, the valve 160 may be configured to be manually opened by the user. The compressed fluid source 130 may include a compressed fluid cannister. In some embodiments, the compressed fluid cannister may be disposed within a recess of the apparel body 110. In some embodiments, the compressed fluid may be rechargeable based on a movement of the user while wearing the apparel device 100. In some embodiments, the compressed fluid cannister may be removeable and replaceable when the compressed fluid of the compressed fluid cannister may be fully discharged. In certain embodiments, a user may connect to a docking station comprising the compressed fluid, where the user is able to receive the compressed fluid from the docking station and/or a compressed fluid source at the docking station.

As further shown in FIG. 2, the apparel device 100 may include the valve 160 in fluid communication with the compressed fluid source 130 and the heat transfer panel 120. The valve 160 may be configured to selectively deliver the compressed fluid from the compressed fluid source 130 to the heat transfer panel 120. In some embodiments, the apparel device 100 may include a controller 150. The valve 160 may include an electronic valve 162 that may be in electronic communication with a controller 150.

The apparel device 100 may further include a sensor 170 in electronic communication with the controller 150. The sensor 170 may include at least one of a temperature sensor and a pressure sensor. The temperature sensor may be configured to detect an internal temperature within an interior of the apparel body 110 and transmit a signal representative of the internal temperature to the controller 150. The controller 150 may be configured to actuate the electronic valve 162 to deliver a flow of the compressed fluid to the heat transfer panel 120 upon the internal temperature exceeding a predetermined temperature. If the internal temperature no longer exceeds the predetermined temperature, the controller 150 may be configured to actuate the electronic valve 162 to cease the flow of the compressed fluid to the heat transfer panel 120. In certain embodiments, the pressure sensor may be configured to detect an amount of the compressed fluid within the compressed fluid source 130.

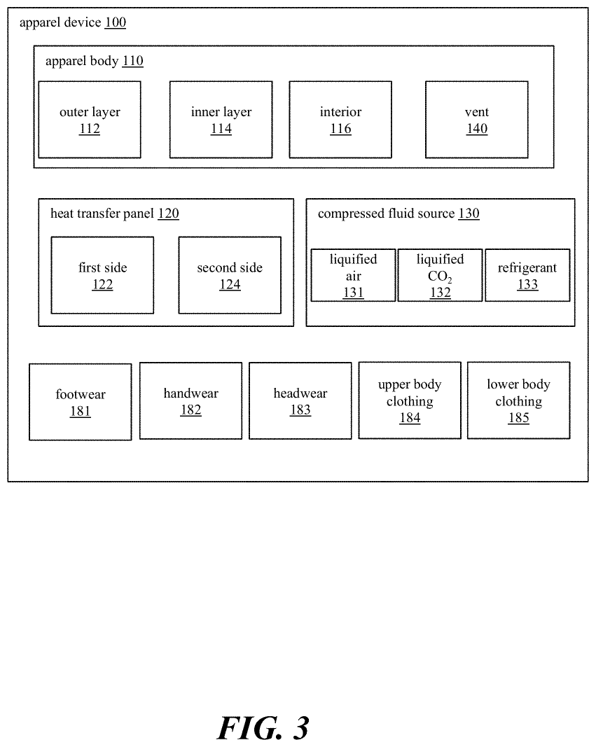

As shown in FIG. 3, in some embodiments, the apparel body 110 may be selected from a group consisting of footwear 181, handwear 182, headwear 183, upper body clothing 184, lower body clothing 185, and combinations thereof. The compressed fluid source 130 may be selected from a group consisting of liquified air 131, liquified carbon dioxide 132, a refrigerant 133, such as R134 and combinations thereof. However, the compressed fluid source 130 may include any appropriately desired compressed fluid source as known in the art.

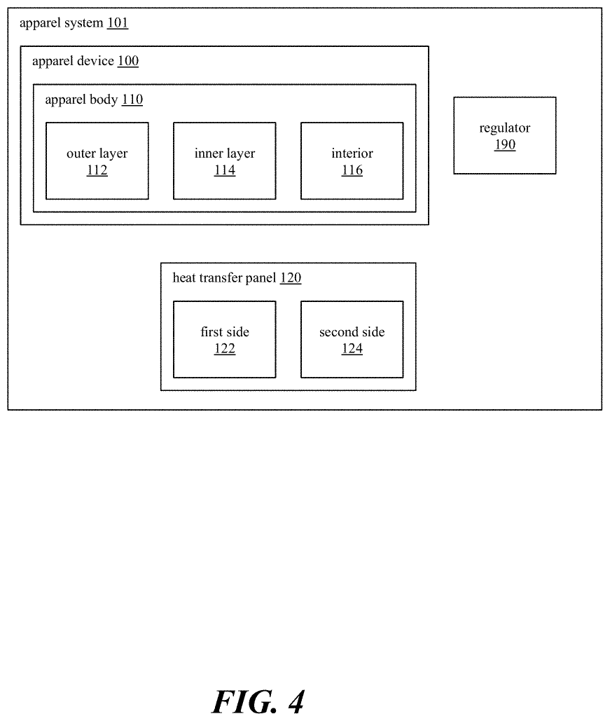

FIG. 4 is a block diagram that describes an apparel system 101, according to some embodiments of the present disclosure. In some embodiments, the apparel system 101 may include an apparel device 100 and a regulator 190 in electronic communication with the apparel device 100. The apparel device 100 may include an apparel body 110. The apparel body 110 may include an outer layer 112 and an inner layer 114. The apparel body 110 may also include an interior 116 configured to receive a body part of a user. A heat transfer panel 120 may be disposed between the outer layer 112 and the inner layer 114.

The regulator 190 may be configured to permit the user to operate the apparel device 100. The heat transfer panel 120 may include a first side 122 and a second side 124. The first side 122 may be disposed adjacent the interior 116 of the apparel body 110. A compressed fluid source 130 may be in fluid communication with the second side 124 of the heat transfer panel 120. The compressed fluid 130 source may be configured to selectively deliver compressed fluid to the heat transfer panel 120. The heat transfer panel 120 may be configured to cool the interior 116 of the apparel body 110 upon delivery of the compressed fluid to the heat transfer panel 120. A vent 140 may disposed within the apparel body 110. The vent 140 may be configured to expel the compressed fluid to an environment outside of the apparel body 110 following the delivery of the compressed fluid to the heat transfer panel 120.

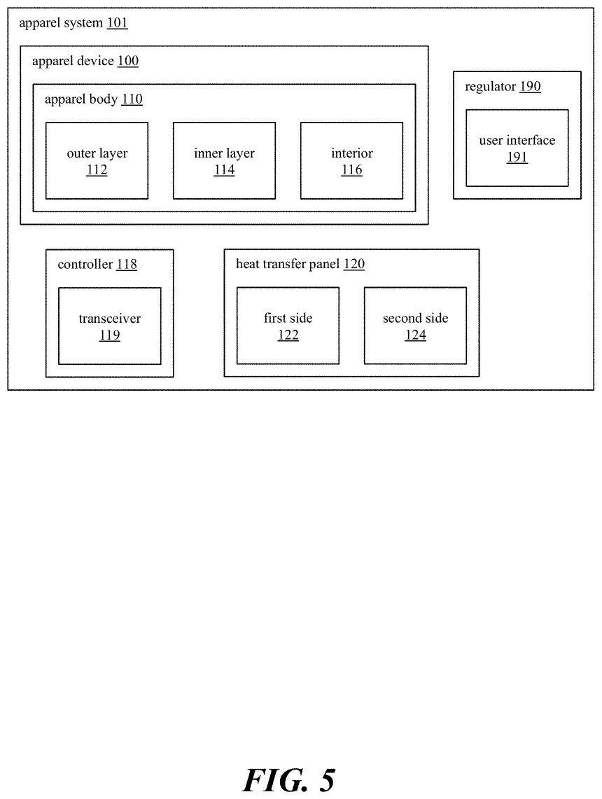

In some embodiments, such as shown in FIG. 5 the apparel system 101 may include a controller 118. The controller 118 may include a transceiver 119 in electronic communication with the regulator 190. In some embodiments, the regulator 190 may include a user interface 191 configured to permit for at least one of a selective delivery and pre-programmed delivery of the compressed fluid to the heat transfer panel 120.

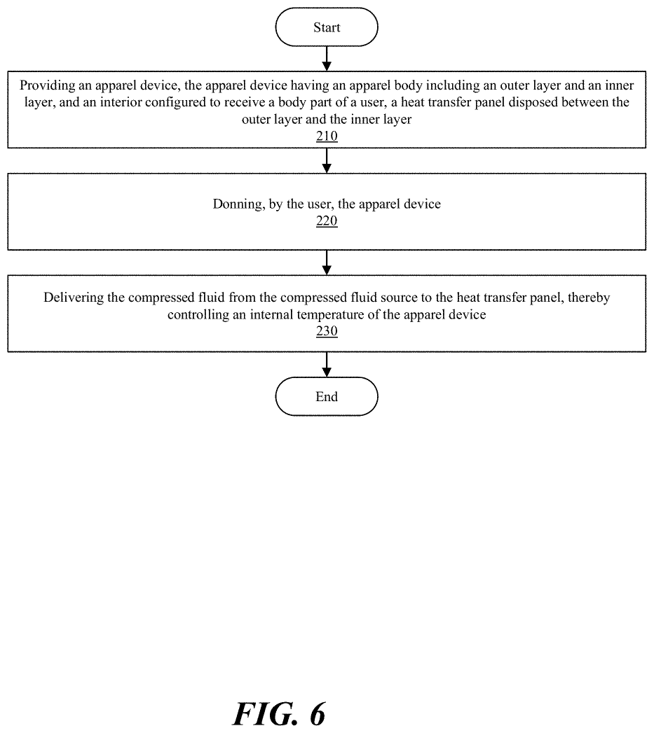

FIG. 6 is a flowchart that describes a method of cooling a body part of a user, according to some embodiments of the present disclosure. In some embodiments, at 210, the apparel method may include providing an apparel device 100, the apparel device 100 having an apparel body 110 including an outer layer 112 and an inner layer 114. An interior 116 may be configured to receive a body part of a user. A heat transfer panel 120 may be disposed between the outer layer 112 and the inner layer. At 220, the apparel method may include donning, by the user, the apparel device 100. At 230, the method may further include delivering the compressed fluid from the compressed fluid source 130 to the heat transfer panel 120, thereby controlling an internal temperature of the apparel device 100 and cooling the body part of the user.

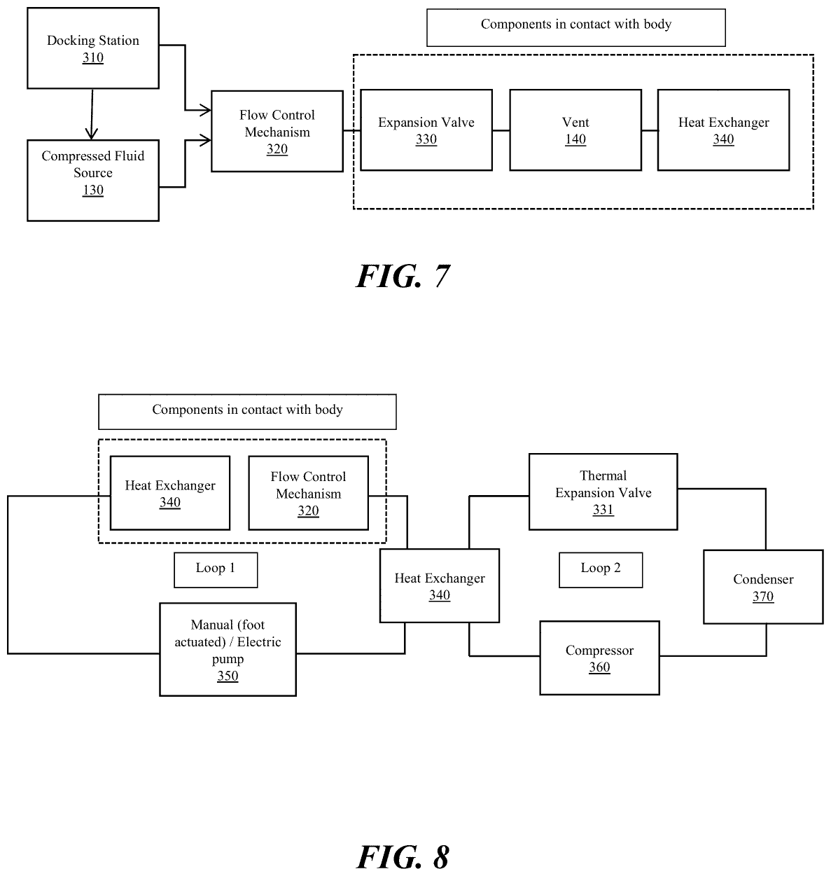

As shown in FIG. 7, a first configuration for a cooling system may utilize an open-loop configuration, whereby compressed fluid may be vented from the system. The first configuration may include a docking station 310 in communication with a flow control mechanism 320. In certain embodiments, the docking station 310 may hold the compressed fluid source 130 and may be in direct communication with the flow control mechanism 320 for delivering the compressed fluid to those components in contact with the body. Alternatively, the compressed fluid source 130 may directly deliver the compressed fluid to those components in contact with the body through the flow control mechanism 320. An expansion valve 330 may meter or otherwise control an amount of the compressed fluid delivered to the heat exchanger 340, such as the heat transfer panel 120, described above. The vent 140 may be configured to expel the compressed fluid to an outside environment. In certain embodiments, mobility of a user may be achieved by utilizing a tank of compressed gas and/or wearing a section of the system and attaching to the docking station 310 for rapid area cooling.

As shown in FIG. 8, a second configuration for the cooling system may utilize a self-contained configuration with a thermally conductive fluid, such as glycol and a refrigerant, such as R134. A loop 1 may include the thermally conductive material, such as glycol, while loop 2 may include the refrigerant. Loop 1 and loop 2 may each comprise a recirculating loop.

Loop 1 may include a pump 350 with a heat exchanger 340 and a flow control mechanism 320 in contact with the body. The heat exchanger 340 may pull heat from the body as the glycol is delivered through the flow control mechanism 320 to the heat exchanger 340. In certain embodiments, the glycol may be circulated through loop 1 by the pump 350. The pump 350 may include a manually activated pump and an electronic pump. In certain embodiments, the pump 350 may be actuated as a user moves or otherwise places pressure against the pump 350. For example, the pump 350 may include a compression pump that circulates the glycol in loop 1.

Loop 2 in connection with loop 1, may include a compressor 360, a condenser 370, a thermal expansion valve 331 and a heat exchanger 340. In certain embodiments, loop 2 may generally describe a vapor-compression cycle, in which the refrigerant may undergo a phase change. A circulating refrigerant may enter the compressor 360 where it is compressed to a high pressure creating a higher temperature and a thermodynamic saturated vapor. The compressed refrigerant as a superheated vapor may pass through the condenser 370 where the refrigerant may be condensed to a liquid. In certain embodiments, rejected heat may be expelled. The condensed refrigerant may pass through the thermal expansion valve 331 where the refrigerant may undergo a reduction in pressure. The pressure reduction may lower the temperature of a liquid and vapor refrigerant mixture. The lower temperature refrigerant may contact the heat exchanger 340, where it may pull heat from the glycol of loop 1 to lower the temperature of the glycol so that the glycol may be delivered to those components in contact with the body of loop 1 to cool the body.

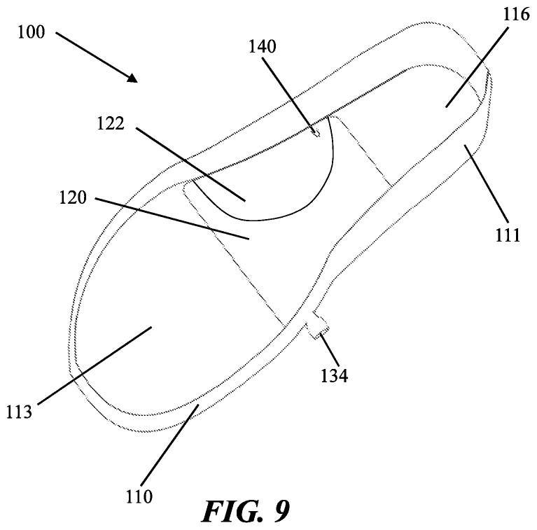

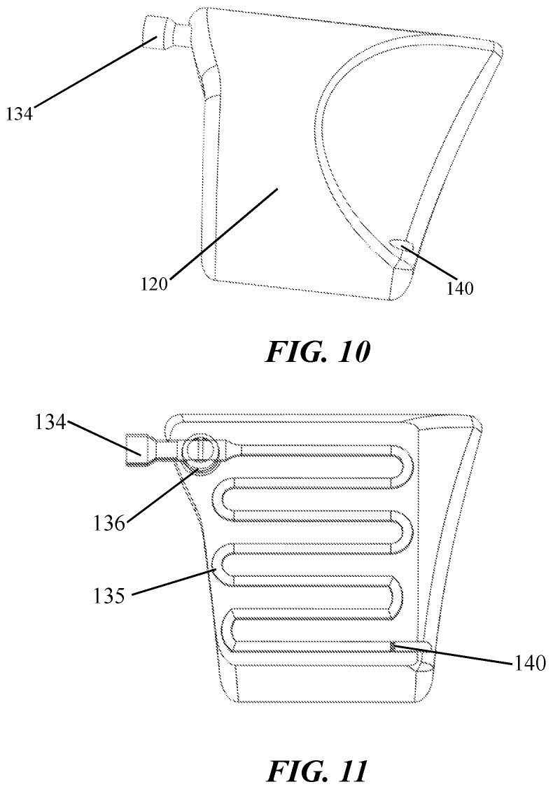

In a particular embodiment, as shown in FIGS. 9-11 the present invention may be embodied in an item of footwear. As shown in FIG. 9-11, the apparel device 100 may include an apparel body 110. The apparel body 110 may include a modular insole 111 that may be configured to be slid or otherwise inserted into an existing footwear item. The modular insole 111 may include a footbed 113. As would be apparent to someone of ordinary skill in the art, the apparel device 100 may be implemented in any appropriately desired item of footwear. For example, the apparel device 100 may include a removable insole, a shoe, a sock, and other appropriately desired footwear items. In addition, as described above, the apparel body 110 may include any appropriately desired apparel body 110. For example, the apparel body may include footwear, handwear, headwear, upper body clothing, lower body clothing, and combinations thereof.

A heat transfer panel 120 may be disposed between an outer layer 112 and an inner layer 114 of the apparel body 110 (FIG. 1). A compressed fluid source 130 (FIG. 1) may connect to an inlet port 134 for the compressed fluid source 130 and be in fluid communication with the second side 124 or underside of the heat transfer panel 120. The first side 122 may be adjacent to the interior 116 of the apparel body 110. In certain embodiments, the first side 122 may be configured to contact an instep or AVA zone on a foot of a user for cooling the user, when implemented within the item of footwear.

A vent 140 may be disposed within the apparel body 110. As described above, an interior 116 of the apparel body may be configured to receive a body part, such as the foot of a user. As shown in FIG. 11, the compressed fluid source 130 may be delivered through a compressed fluid path 135, such that the compressed fluid source 130 is in fluid communication with the second side 124 of the heat transfer panel 120 to cool the interior 116 of the apparel body 110. The compressed fluid path 135 may include a mechanism such as a perforated capillary tube, a series of perforated capillary tubes or other appropriately desired delivery mechanisms for delivering the compressed fluid to the second side 124 of the heat transfer panel 120. In certain embodiments, the inlet port 134 may include an adjustable needle valve which and a flow of the compressed fluid may be adjusted by turning a screw 136.

### Examples

The twelve tables presented below include data from twelve separate tests. Each test was completed using a temperature plate with a heating source directed to a first side of a heating plate and using compressed carbon dioxide as a compressed liquid cooling source directed to a second side of the heating plate, where the second side of the heating plate is opposite the first side of the heating plate. A 50 lb compressed carbon dioxide tank with a needle valve, tubing, and nozzle were used to direct the compressed liquid cooling source to the second side of the heating plate.

A flow from the tube denotes shutting off a supply of the compressed carbon dioxide from the compressed liquid cooling source and only using the carbon dioxide within a coil of the tubing. A flow from the carbon dioxide tank denotes leaving the valve of the tank open and having a continuous delivery of carbon dioxide from the carbon dioxide tank. The flow control settings ranged from 0-2, which involved rotating the needle valve a 1/10 rotation at the temperature plate. A length of the coil was 103.5 inches, an inner diameter of the coil was 0.28 inches, an inner area of the coil was 0.06158 square inches, and an inner volume of the tube coil was 6.373 inches cubed.

| | |
|---|---|
| Test 1 | Control |

| Time | Temp A | Temp B |
|---|---|---|
| 0 | 47 | 40 |
| 1 | 38 | 35 |
| 2 | 34 | 32 |
| 3 | 31 | 28 |
| 4 | 28 | 27 |
| 5 | 26 | 25 |
| 6 | 23 | 22 |
| 7 | 22 | 20 |
| 8 | 20 | 18 |
| 9 | 18 | 17 |
| 10 | 17 | 16 |
| 11 | 16 | 15 |
| 12 | 15 | 14 |
| 13 | 14 | 13 |
| 14 | 14 | 12 |
| 15 | 13 | 11 |
| 16 | 12 | 11 |
| 17 | 12 | 10 |
| 18 | 12 | 10 |
| 19 | 11 | 9 |
| 20 | 11 | 9 |

| | |
|---|---|
| Test 2 | Control |

| Time | Temp A | Temp B |
|---|---|---|
| 0 | 39 | 37 |
| 1 | 38 | 36 |
| 2 | 35 | 34 |
| 3 | 32 | 30 |
| 4 | 29 | 29 |
| 5 | 27 | 27 |
| 6 | 25 | 24 |
| 7 | 23 | 22 |
| 8 | 21 | 20 |
| 9 | 19 | 18 |
| 10 | 18 | 17 |
| 11 | 17 | 16 |
| 12 | 16 | 15 |
| 13 | 15 | 14 |
| 14 | 14 | 13 |
| 15 | 14 | 13 |
| 16 | 13 | 12 |
| 17 | 12 | 11 |
| 18 | 12 | 11 |
| 19 | 11 | 11 |
| 20 | 11 | 10 |

| | |
|---|---|
| Test 3 | Flow from Bottle |

| Time | Temp A | Temp B |
|---|---|---|
| 0 | 42 | 39 |
| 1 | 28 | 17 |
| 2 | 22 | 14 |
| 3 | 18 | 12 |
| 4 | 15 | 10 |
| 5 | 13 | 9 |

*Note:
42 SCFM

| | |
|---|---|
| Test 4 | Flow from loop |

| Time | Temp A | Temp B |
|---|---|---|
| 0 | 42 | 37 |
| 1 | 30 | 19 |
| 2 | 27 | 26 |

* Note:
Flow Setting 2

| | |
|---|---|
| Test 5 | Flow from loop |

| Time | Temp A | Temp B |
|---|---|---|
| 0 | 35 | 33 |
| 1 | 28 | 28 |
| 2 | 26 | 27 |

* Note:
Flow Setting 1

| | |
|---|---|
| Test 6 | Flow from Loop |

| Time | Temp A | Temp B |
|---|---|---|
| 0 | 44 | 37 |
| 1 | 30 | 19 |
| 2 | 27 | 20 |
| 3 | 25 | 21 |
| 4 | 23 | 21 |

* Note:
Flow Setting 1

| | |
|---|---|
| Test 7 | Flow from Loop |

| Time | Temp A | Temp B |
|---|---|---|
| 0 | 41 | 38 |
| 1 | 35 | 26 |
| 2 | 30 | 22 |
| 3 | 27 | 20 |
| 4 | 26 | 18 |

* Note:
Flow setting 1.25

| | |
|---|---|
| Test 8 | Flow from Loop |

| Time | Temp A | Temp B |
|---|---|---|
| 0 | 43 | 39 |
| 1 | 33 | 23 |
| 2 | 30 | 24 |

* Note:
Flow setting 1.5

| | |
|---|---|
| Test 9 | Flow from Loop |

| Time | Temp A | Temp B |
|---|---|---|
| 0 | 43 | 39 |
| 1 | 33 | 24 |
| 2 | 30 | 23 |

* Note:
Flow setting 1.5

| | |
|---|---|
| Test 10 | Flow from Loop |

| Time | Temp A | Temp B |
|---|---|---|
| 0 | 43 | 39 |
| 1 | 40 | 32 |
| 2 | 36 | 26 |

* Note:
Short Bursts

| | |
|---|---|
| Test 11 | Flow from Tank |

| Time | Temp A | Temp B |
|---|---|---|
| 0 | 43 | 39 |
| 30 | 35 | 19 |
| 60 | 28 | 12 |
| 90 | 22 | 6 |
| 120 | 17 | 0 |

* Note:
Flow setting 1

| | |
|---|---|
| Test 12 | Flow from Tank |

| Time | Temp A | Temp B |
|---|---|---|
| 0 | 45 | 39 |
| 30 | 29 | 9 |
| 60 | 17 | −4 |
| 90 | 7 | −13 |

When flowing from the carbon dioxide tank at 500 pounds per square inch (psi) and restricting the compressed fluid flow at the needle valve closest to the heating plate (setting 1), it took 60 seconds to cool a 2×3 inch metal plate heated to 105 degrees Celsius to a target temperature of 55 degrees Celsius. When flowing from the coil starting at 500 psi and restricting the compressed fluid flow at the need valve closest to the heating plate (setting 1) it took 60 seconds to cool a 2×3 inch metal plate heated to 105 degrees Celsius to a temperature of 66 degrees Celsius. The expansion cooling effect was seen more efficiently in the first minute of the compressed carbon dioxide flowing from the coil as the compressed carbon dioxide expanded from 500 psi to 250 psi. Cooling diminished after an initial expansion.

Advantageously, the control tests showed that at an ambient environmental temperature, the heating plate may be cooled at a rate of 2 degrees Celsius per minute. The tested cooling rate stayed consistent throughout tests 3-12. The testing has shown that where a compressed liquid cooling source is delivered to the heating plate, it is able to draw heat from the heating plate, such as to effectively cool the heating plate.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. An apparel device, comprising:
an apparel body including an outer layer and an inner layer, and an interior configured to receive a body part of a user;
a heat transfer panel disposed between the outer layer and the inner layer, wherein the heat transfer panel includes a first side and a second side, the first side disposed adjacent the interior of the apparel body;
a compressed fluid source in fluid communication with the second side of the heat transfer panel, wherein the compressed fluid source is configured to selectively deliver compressed fluid to the heat transfer panel, and wherein the heat transfer panel is configured to cool the interior of the apparel body upon delivery of the compressed fluid to the heat transfer panel; and
a vent disposed within the apparel body, wherein the vent is configured to expel the compressed fluid to an environment outside of the apparel body following the delivery of the compressed fluid to the heat transfer panel.

2. The apparel device of claim 1, further comprising a valve in fluid communication with the compressed fluid source and the heat transfer panel, wherein the valve is configured to selectively deliver the compressed fluid from the compressed fluid source to the heat transfer panel.

3. The apparel device of claim 2, wherein the valve is a mechanical valve and is automatically opened when a predetermined amount of pressure is applied to the apparel body.

4. The apparel device of claim 2, wherein the valve is configured to be manually opened by the user.

5. The apparel device of claim 2, further comprising a controller, wherein the valve includes an electronic valve that is in electronic communication with the controller.

6. The apparel device of claim 5, further comprising a sensor in electronic communication with the controller.

7. The apparel device of claim 6, wherein the sensor comprises at least one of a temperature sensor and a pressure sensor, the temperature sensor configured to detect an internal temperature within an interior of the apparel body and transmit a signal representative of the internal temperature to the controller, wherein the controller is configured to actuate the electronic valve to deliver a flow of the compressed fluid to the heat transfer panel upon the internal temperature exceeding a predetermined temperature.

8. The apparel device of claim 7, wherein once the internal temperature no longer exceeds the predetermined temperature, the controller is configured to actuate the electronic valve to cease the flow of the compressed fluid to the heat transfer panel upon the internal temperature not exceeding the predetermined temperature.

9. The apparel device of claim 7, wherein the pressure sensor is configured to detect an amount of the compressed fluid within the compressed fluid source.

10. The apparel device of claim 1, wherein the compressed fluid source includes a compressed fluid cannister.

11. The apparel device of claim 10, wherein the compressed fluid cannister is disposed within a recess of the apparel body.

12. The apparel device of claim 10, wherein the compressed fluid is rechargeable based on a movement of the user while wearing the apparel device.

13. The apparel device of claim 10, wherein the compressed fluid cannister is removeable and replaceable when the compressed fluid of the compressed fluid cannister is fully discharged.

14. The apparel device of claim 1, wherein the apparel body is selected from a group consisting of footwear, handwear, headwear, upper body clothing, lower body clothing, and combinations thereof.

15. The apparel device of claim 1, wherein the compressed fluid is selected from a group consisting of liquified air, liquified carbon dioxide, a refrigerant, and combinations thereof.

16. An apparel system for cooling a body part of a user, comprising:
an apparel device having an apparel body including an outer layer and an inner layer, and an interior configured to receive the body part of the user, a heat transfer panel disposed between the outer layer and the inner layer, wherein the heat transfer panel includes a first side and a second side, the first side disposed adjacent the interior of the apparel body, a compressed fluid source in fluid communication with the second side of the heat transfer panel, wherein the compressed fluid source is configured to selectively deliver compressed fluid to the heat transfer panel, and wherein the heat transfer panel is configured to cool the interior of the apparel body upon delivery of the compressed fluid to the heat transfer panel, and a vent disposed within the apparel body, wherein the vent is configured to expel the compressed fluid to an environment outside of the apparel body following the delivery of the compressed fluid to the heat transfer panel; and
a regulator in electronic communication with the apparel device, wherein the regulator is configured to permit the user to operate the apparel device.

17. The apparel system of claim 16, further comprising a controller, wherein the controller includes a transceiver in electronic communication with the regulator.

18. The apparel system of claim 17, wherein the regulator includes a user interface configured to permit for at least one of a selective delivery and pre-programmed delivery of the compressed fluid to the heat transfer panel.

19. A method of cooling a body part of a user, comprising steps of:
providing an apparel device, the apparel device having an apparel body including an outer layer and an inner layer, and an interior configured to receive the body part of the user, a heat transfer panel disposed between the outer layer and the inner layer, wherein the heat transfer panel includes a first side and a second side, the first side disposed adjacent the interior of the apparel body, a compressed fluid source in fluid communication with the second side of the heat transfer panel, wherein the compressed fluid source is configured to selectively deliver compressed fluid to the heat transfer panel, and wherein the heat transfer panel is configured to cool the interior of the apparel body upon delivery of the compressed fluid to the heat transfer panel, and a vent disposed within the apparel body, wherein the vent is configured to expel the compressed fluid to an environment outside of the apparel body following the delivery of the compressed fluid to the heat transfer panel;
donning, by the user, the apparel device; and
delivering the compressed fluid from the compressed fluid source to the heat transfer panel, thereby controlling an internal temperature of the apparel device and cooling the body part of the user.

20. The method of claim 19, further comprising a step of replacing the compressed fluid source upon the compressed fluid source being fully discharged.
